(19) United States
(12) Patent Application Publication
(10) Pub. No.: US 2007/0246438 A1
(43) Pub. Date: Oct. 25, 2007

(54) Medication keycode lock

(76) Inventor: Ching-Chang Chen, Taipei City (TW)

Correspondence Address: TROXELL LAW OFFICE PLLC, SUITE 1404, 5205 LEESBURG PIKE, FALLS CHURCH, VA, 22041, US

(21) Appl. No.: 11/404,083

(22) Filed: Apr. 14, 2006

(51) Int. Cl. B65D 51/24; B65D 51/00; A61B 19/00

(52) U.S. Cl. 215/302; 604/415; 215/247

(57) ABSTRACT

A medication keycode lock fan light fabricated from a sealing cap, a keycode cover and a keycode cover opener. A screw opening is formed in the sealing cap corresponding to position of an elastic septum of the medicine bottle. The keycode cover screws into the screw opening of the sealing cover, and is used to cover the elastic septum of the medicine bottle. Furthermore, a keycode hole having a set profile is defined in a surface of the keycode cover, and an end of the keycode cover opener is configured with a key that matches the keycode hole. The key of the keycode cover opener and the keycode hole must be in exact corresponding juxtaposition to enable unscrewing of the keycode cover, thereby uncovering the elastic septum of the medicine bottle and effectively resolving the problem of using the wrong medication due to inadvertence of the person concerned.

### BACKGROUND OF THE INVENTION

(a) Field of the Invention

The present invention relates to a foolproof technology applied to prevent administering wrong medications by injection, and more particularly provides a medication keycode lock that effectively resolves the problem of using the wrong medication due to inadvertence of the person concerned.

(b) Description of the Prior Art

Many of the medications in the majority of current medical and health care plans or courses of medical treatment must be administered by injection using syringes to enable the medication to enter the human body and take affect. Moreover, the majority of medications administered by injection are contained in a special medicine bottle 10, as depicted in FIG. 1, and medicine bottles similar to the medicine bottle 10 are fitted with an elastic septum 11 that is used to seal an opening of the medicine bottle 10, thereby preventing leakage or deterioration of the medication contained in the medicine bottle 10.

In use, a needle 21 of a hypodermic syringe 20 is made to pierce the elastic septum 11 and penetrate deeply into the medication of the medicine bottle 10, whereupon the medication is sucked into the hypodermic syringe 20. Hence, the entire procedure for administering the medication for medical treatment is certainly convenient. However, shape, size and method of labeling the conventional medicine bottles 10 used to contain injection medications are almost similar, and a person is apt to take a wrong medicine bottle if the label is not first carefully identified, in addition, the elastic septum 11 of the medicine bottle 10 is normally exposed, which is apt to result in a person inadvertently using the wrong medication causing irreparable regret.

### SUMMARY OF THE INVENTION

In light of the aforementioned shortcomings, the present invention assembles a sealing cover, a keycode cover and a keycode cover opener to fabricate a medication keycode lock that effectively resolves the problem of using the wrong medication due to inadvertence of the person concerned. The sealing cover seals tight an opening of a medicine bottle, and a screw opening is formed in the sealing cover corresponding to position of an elastic septum of the medicine bottle. The keycode cover screws into the screw opening of the sealing cover, and is used to cover the elastic septum of the medicine bottle.

Furthermore, a keycode hole having a set profile is defined in a surface of the keycode cover, and an end of the keycode cover opener is configured with a key that matches the keycode hole. The key of the keycode cover opener and the keycode hole must be in exact corresponding juxtaposition to enable unscrewing of the keycode cover, thereby uncovering the elastic septum of the medicine bottle and effectively resolving the problem of using the wrong medication due to inadvertence of the person concerned.

To enable a further understanding of said objectives and the technological methods of the invention herein, brief description of the drawings is provided below followed by detailed description of the preferred embodiments.

### BRIEF DESCRIPTION OF THE DRAWINGS

FIG. 1 shows a schematic view of a prior art medicine bottle during usage.

FIG. 2 shows an exploded structural view of a keycode lock according to the present invention.

FIG. 3 shows a schematic view of a keycode cover opener of the present invention in use.

FIG. 4 shows a schematic view of the keycode cover opener mounted in a keycode cover according to the present invention.

FIG. 5 shows a schematic view of the keycode cover detached from a medicine bottle according to the present invention.

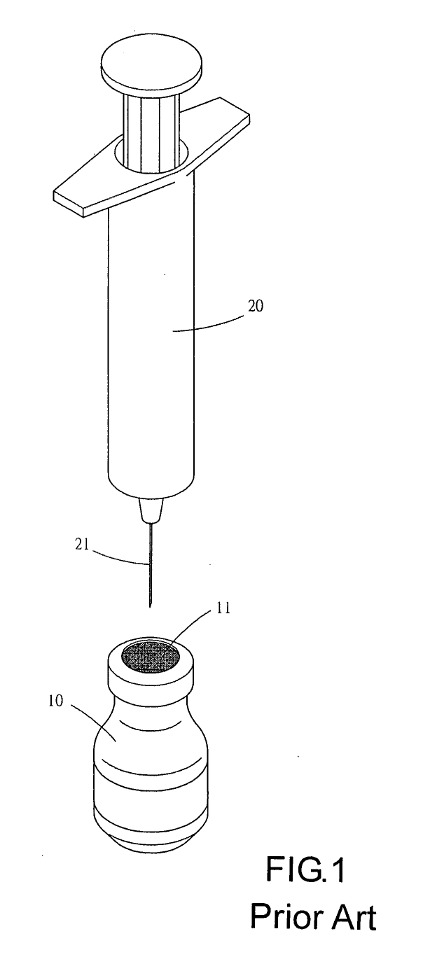

### DETAILED DESCRIPTION OF THE PREFERRED EMBODIMENTS

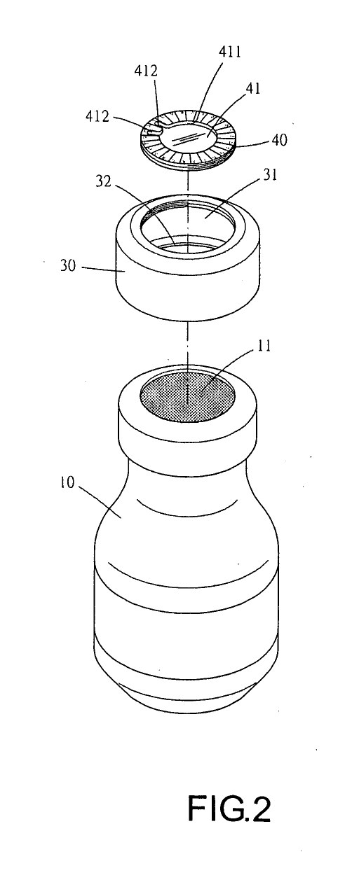

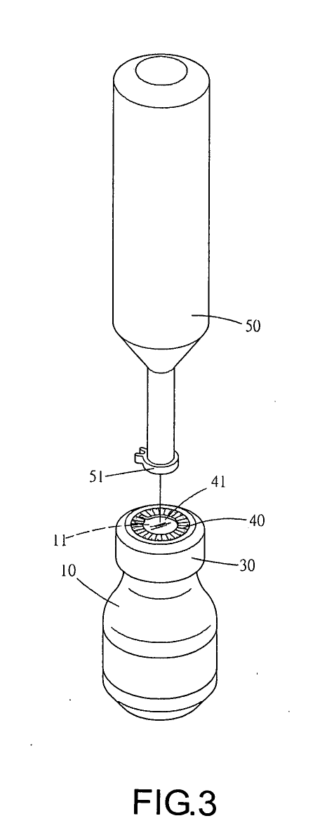

Referring to FIGS. 2 and 3, which show a medication keycode lock of the present invention, structured to comprise a sealing cap 30, a keycode cover 40 and a keycode cover opener 50, wherein the sealing cap 30 seals tight an opening of a medicine bottle 10, and a screw opening 31 is formed in the sealing cap 30 corresponding to position of an elastic septum 11 of the medicine bottle 10. The keycode cover 40 screws into the screw opening 31 of the sealing cap 30, and is used to cover the elastic septum 11 of the medicine bottle 10.

In an embodiment of the present invention, a bottom portion of the sealing cap 30 can be configured with a bottleneck connection point 32 as a ring form structural body, which in conjunction with a neck of the medicine bottle 10 forms a fastening function that enables firm disposition at an opening of the medicine bottle 10. Furthermore, a keycode hole 41 having a set profile is defined in a surface of the keycode cover 40. Form of the keycode hole 41 profile is set according to the medication contained in the medicine bottle 10, that is, profile form of the keycode cover 40 disposed in the opening is dissimilar for each medicine bottle 10 containing different medications.

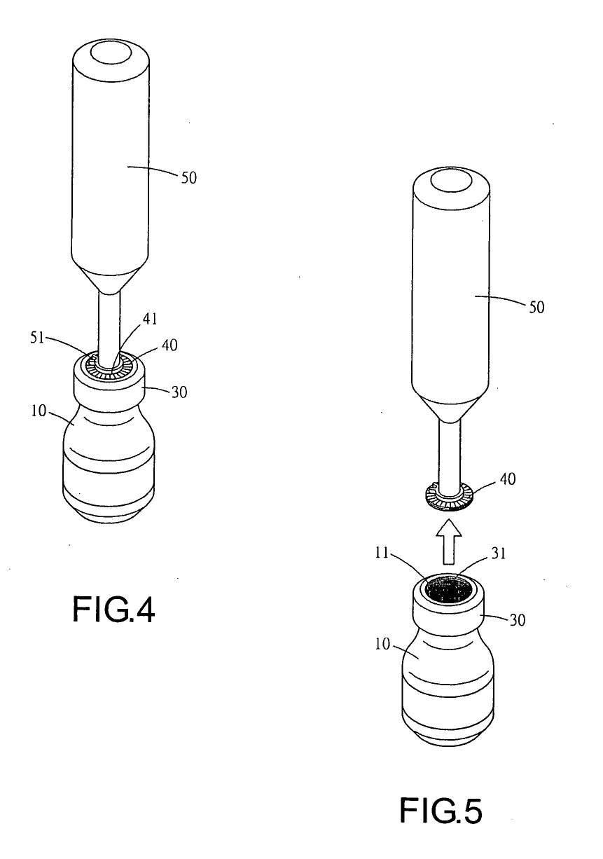

The keycode cover opener 50 is configured to match the keycode hole 41 of set profile form, and each keycode cover opener 50 is configured with a key 51 that matches the keycode hole 41. When a medication is needed, the correct keycode cover opener 50 must be obtained (that is, the keycode cover opener 50 having the key 51 that matches the keycode hole 41). Moreover, referring to FIGS. 4 and 5, the key 51 of the keycode cover opener 50 and the keycode hole 41 must be in exact corresponding juxtaposition to enable unscrewing of the keycode cover 40, thereby uncovering the elastic septum 11 of the medicine bottle 10 in order to implement follow-up injection procedure.

Actual injection procedure involves first obtaining the medicine bottle 10, then an opening action of the keycode cap 30 must be carried out before a hypodermic syringe can be used to extract the medication contained in the medicine bottle 10. Moreover, the keycode cap 30 must be unscrewed using the correct keycode cover opener 50, thereby providing a second confirmation action to verify that the medication contained in the medicine bottle 10 is the correct one, which effectively resolves the problem of using the wrong medication due to inadvertence of the person concerned.

Furthermore, the keycode hole 41 of the keycode cover 40 is provided with a base hole 411, an inner ring of which is configured with a set number of indentations 412, which in conjunction with differences of included angles are used to set the profile form of the keycode hole 41. It can also be appreciated that marks or symbols can be mounted on a periphery of the keycode hole 41 to facilitate identifying position or name of the indentations 412. Moreover, position of the symbols on the indentations 412 can be additionally marked on the medicine bottle 10 and the keycode cover opener 50, thereby enabling a user to know what is contained in the medicine bottle 10 receptacle based on the symbols marked on the medicine bottle 10, and thus facilitating the user in locating the correct keycode cover opener 50 to open the keycode cover 40.

According to the aforementioned, the present invention provides a preferable and feasible structure for a medication keycode lock. Accordingly, a new patent application is proposed herein.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. A medication keycode lock comprising:
a sealing cap that seals tight an opening of a medicine bottle, and a screw opening is formed in the sealing cover corresponding to position of an elastic septum of the medicine bottle;
a keycode cover that screws into the screw opening of the sealing cap, and is used to cover the elastic septum of the medicine bottle;
a keycode cover opener that is used to unscrew the keycode cover.

2. The medication keycode lock according to claim 1, wherein a keycode hole having a set profile is defined in a surface of the keycode cover, and an end of the keycode cover opener is configured with a key that matches with the keycode hole.

3. The medication keycode lock according to claim 1, wherein a bottom portion of the sealing cap is configured with a bottleneck connection point as a ring form structural body, and the bottleneck connection point in conjunction with a neck of the medicine bottle forms a fastening function.

4. The medication keycode lock according to claim 1, wherein the keycode hole is provided with a base hole, an inner ring of which is configured with a set number of indentations, and differences in number, shape, included angle, and so on, of the indentations are used to set profile form of the keycode hole.

5. The medication keycode lock according to claim 3, wherein marks, symbols or numbers are mounted on a periphery of the keycode hole to facilitate identifying position or name of the indentations.
